(19) United States
(12) Patent Application Publication
(10) Pub. No.: US 2005/0283162 A1
(43) Pub. Date: Dec. 22, 2005

(54) Intraocular lens inserter

(76) Inventor: Byron A. Stratas, Wilmington, NC (US)

Correspondence Address: WILLIAM J. MASON;MACCORD MASON PLLC, POST OFFICE BOX 1489, WRIGHTSVILLE BEACH, NC, 28480, US

(21) Appl. No.: 10/874,043

(22) Filed: Jun. 22, 2004

(51) Int. Cl. A61F 9/00

(52) U.S. Cl. 606/107

(57) ABSTRACT

An intraocular lens is laterally compressed prior to insertion through an eye incision using an inserter adapted to minimize damage to the intraocular lens. The inserter includes a handpiece with a longitudinal bore, a cartridge holder at the distal end of the handpiece bore, and a discharge nozzle with a tapered bore on the distal side of the cartridge holder. A prepackaged cartridge holding an intraocular lens is mounted in the cartridge holder. The intraocular lens has an optic with leading and trailing haptics axially aligned with the handpiece and nozzle bores. A plunger compresses the lens by moving the lens through the nozzle tapered bore. One plunger described includes flexible distal arms that compress inwardly to maintain the arm tips against the sides of the optic, while another plunger includes an angular tip that engages a hole in the leading haptic to pull the lens through the tapered bore.

### BACKGROUND OF THE INVENTION

(1) Field of the Invention

The present invention relates to an intraocular lens insertion instrument and to an intraocular lens, and in particular to an improved inserter and lens permitting accurate lens insertion with minimal risk of damage to the lens.

(2) Description of the Prior Art

During cataract surgery, the defective natural lens is removed by inserting a cutting instrument into the lens through a small (1-4 mm) incision in the ocular tissue of the eye. Ultrasonic vibration of the instrument emulsifies the natural lens into minute particles that are then aspirated from the eye. The natural lens is then replaced with an artificial intraocular lens (IOL), which is inserted through the incision. Some intraocular lenses for refractive correction of an eye are inserted by a similar procedure, but without removal of the natural lens.

An IOL is comprised of a central optic, which serves as the natural lens replacement and projections on opposite sides of the optic, known as haptics that are used to anchor the IOL in the desired position in the eye. The IOL normally has a lateral diameter of at least 6 mm and a thickness of 1-2 mm. In order to insert the IOL without enlarging the incision, with a resultant increase in trauma and risk of infection, IOLs are constructed of a flexible material, commonly a silicone or acrylic polymeric material that can be laterally compressed to the desired dimension. After insertion, the compressive force on the lens is released, allowing the lens to resume its uncompressed configuration. The surgeon then positions the lens as required.

An insertion instrument, or inserter, is normally used to compress and insert the IOL. Such instruments are described in U.S. Pat. No. 4,681,102 to Bartell, and in numerous subsequent patents purporting to describe improvements in the Bartell design. Generally, an IOL insertion instrument is comprised of a handpiece that includes an elongated lumen or bore with proximal and distal ends. A plunger is positioned to slide through the bore from a retracted to an inserted position. The plunger has an enlarged proximal end to be engaged by the surgeon, and a tip at its distal end that is used to contact the IOL. An IOL cartridge is positioned in front of the bore distal end so that an IOL held within the cartridge is engaged by the plunger tip as the plunger is moved toward its extended position. An elongated discharge nozzle is axially aligned on the distal side of the cartridge. This nozzle has a tapered bore with a cross-sectional diameter at its proximal end approximately equal to the lateral diameter of the lens as the lens is mounted in the cartridge, and tapers to the desired compressed diameter at its distal end. The distal end of the tip is of a diameter sufficient to permit insertion into the slit in the eye.

During the operation, an IOL is mounted into a cartridge. A lubricant is normally introduced into the area where the lens is to be compressed to the insertion diameter. The cartridge is then mounted in the inserter cartridge holder with the IOL oriented so that the haptics are axially aligned, i.e., one haptic is in front of the lens and one haptic is behind the lens. While the IOL may be in an uncompressed state within the cartridge chamber, the IOL is usually at least partially compressed into a “U” or “W” shape by closure of cartridge or the use of a ram to deform the IOL. Alternatively, the IOL may be prepackaged in a cartridge, with the lubricant added by the physician when the cartridge is to be mounted in the holder.

The plunger is moved toward its extended position, so that the plunger tip engages the rear of the IOL pushing the IOL forward into the tapered bore of the discharge nozzle. As the IOL moves through the tapered bore, the IOL is laterally compressed until its diameter is sufficiently small to allow discharge from the nozzle discharge opening. The end of the nozzle is then inserted through the eye slit and the plunger is moved to its fully extended position to discharge the IOL from the nozzle into the eye. Following discharge, the IOL expands to its original shape and dimension.

Compressible IOLs are necessarily constructed of a soft material that is easily scratched, torn or otherwise damaged. Moreover, the outwardly extending haptics are susceptible to tearing, twisting or permanent deformation during compression of the IOL. Therefore, considerable care must be exercised during IOL compression and insertion. While the prior art has proposed various instrument constructions, including plunger tip constructions, to address this concern, there is still a need for an IOL insertion instrument that is capable of accurately compressing and inserting an intraocular lens without a significant risk of damaging the lens due to contact with the plunger tip or improper orientation of the lens during compression and insertion. There is also a need for an intraocular lens adapted for use with the improved inserter.

### SUMMARY OF THE INVENTION

Generally, the present invention is directed to an improved intraocular lens inserter, and in particular to an inserter having a plunger tip that minimizes the risk of damage to the intraocular lens while permitting accurate compression and insertion of the lens. In one embodiment of the invention, an improved lens is also described for use with the improved inserter.

The present inserter is similar in some respects to prior art intraocular lens inserters. That is, the present inserter is comprised of a handpiece with an elongated lumen or bore having proximal and distal ends, and a plunger that is slidable within the bore between a retracted position and an inserted position. A cartridge with a prepackaged IOL, to be described in greater detail, is positioned in a cartridge holder at the distal end of the bore so that the IOL is held in axial alignment with the handpiece bore for engagement by the distal end of the plunger. The IOL is positioned in the cartridge with one haptic on the back or trailing side of the optic and a second haptic on the front or leading side of the optic. A discharge nozzle with a bore that tapers inwardly toward a distal tip is positioned on the distal side of the cartridge to receive an IOL pushed from the cartridge by the plunger.

The present inserter differs, however, in several respects, and in particular in the configuration of the plunger. In one embodiment of the invention, the inserter plunger has a forked forward section, i.e., the distal end of the plunger is split into two resilient elongated arms that are separated from each other at their distal ends so that the tips of the arms expand outwardly to engage the handpiece bore wall when the plunger is in its retracted position. When the plunger is moved toward its fully inserted position, the tips of the arms ride in sequence along the walls of the handpiece bore, the cartridge chamber, and finally the tapered bore of the discharge nozzle where the tips are urged toward each other as the IOL is laterally compressed by the tapered bore wall. The length of the arms is preferably at least equal to the length of the trailing haptic, and more preferably equal to up to the length of the longitudinal length of the entire IOL.

Moreover, the inserter plunger of this embodiment of the invention has an upper side and a lower side, with the lower side of the elongated segments being cut away to provide a space between the bore wall and the lower surfaces of the elongated segments. Preferably, about one-half of each segment is cut away at a distance at least equal to the length of the trailing haptic.

Thus, taken together, the inserter of a preferred embodiment of the invention is comprised of a handpiece with a longitudinal bore, a prepackaged cartridge holder at the distal end of the handpiece bore having an IOL cartridge chamber in alignment with the handpiece bore, a discharge nozzle on the opposite side of the cartridge holder having a tapered bore aligned with the handpiece bore, and a plunger of the above description having tips moveable from the handpiece bore through the IOL cartridge chamber and the tapered bore.

Specifically, the handpiece includes a body section with a bore having a longitudinal axis, a given cross-sectional diameter, a proximal end and a distal end. The cartridge holder at the distal end of the handpiece and the discharge nozzle on the opposite side of the cartridge holder may be integrally formed.

A cartridge holding a prepackaged IOL is sized to fit into the cartridge holder. The cartridge includes an outer housing enclosing an IOL chamber having a longitudinal axis aligned with the longitudinal axes of the handpiece bore and the tapered bore when mounted in the cartridge holder. The cartridge includes a proximal opening positionable adjacent the handpiece bore distal opening and a distal opening positionable adjacent the proximal opening of the tapered bore. The diameters of the handpiece bore, the chamber and the proximal end of the tapered bore are approximately equal, providing a uniform wall diameter. The cartridge chamber is adapted to hold an IOL with the optic aligned with the longitudinal axis of the bore, and with one haptic on the trailing side of the optic and another haptic on the leading side.

The IOL is preferably in an uncompressed state within the cartridge, with the lateral diameter of the optic when the IOL is packaged in the cartridge chamber being approximately equal to the cross-sectional diameter of the cartridge chamber. However, the IOL may alternatively be packaged in a partially compressed state, e.g., the IOL may be cupped into a “U” shape. Also, the IOL may be in an uncompressed state until the IOL is to be inserted, and then laterally compressed to reduce the cross-sectional diameter of the IOL, or parts thereof. For example, the cartridge in which the IOL is enclosed may include laterally compressible side walls that press against parts of the IOL when the cartridge is laterally compressed. In a preferred use, the haptic anchor wires are pressed inwardly and over the central part of the haptics by lateral compression of the cartridge, thereby minimizing the risk of damage to the anchor wires.

The IOL may be packaged in the cartridge with a lubricant such as a viscoelastic, e.g., Healon, or the cartridge can include one or more injection ports in a wall of the cartridge for injection of a lubricant into the chamber housing the IOL immediately prior to use.

When the plunger is pushed from its retracted position toward its inserted position, the plunger arm tips ride along the walls of the handpiece bore and then the cartridge chamber until the tips engage the outer edges of the optic within the cartridge chamber. Contact of the tips with the trailing haptic is avoided by the presence of the cutout areas on the lower surfaces of the segments. Instead, the segments slide over the haptic, which then rests beneath the segments as the IOL is moved forward.

As the IOL is pushed from the cartridge chamber into the tapered bore of the discharge nozzle, the sides of the IOL cup upward into a “U” shape due to narrowing of the bore diameter. At the same time, the plunger arm tips are pressed inwardly by the tapered bore wall. Thus, the plunger tips remain against the sides of the IOL optic, providing a uniform pressure on both sides of the optic as the bore diameter decreases and the IOL is laterally compressed. Upon reaching the distal end of the inserter tip section, the IOL is fully compressed and the tips are in their fully compressed state.

A discharge nozzle with a tapered bore is positioned on the distal side of the cartridge to receive the IOL. In a preferred embodiment, the discharge nozzle bore includes rifling or grooves to guide the haptics upwardly as the IOL is compressed, thereby aiding in compression, as well as protecting the haptics or parts thereof, such as the haptic anchor wires. These grooves may extend from the proximal to the distal end of the nozzle bore, and may be inclined upwardly to guide the leading haptic edges, or the anchor wires thereon, upwardly as the IOL is pushed forward.

When the plunger is in its fully inserted position, the IOL is discharged from the distal end of the nozzle, and the tips are in their fully compressed state, projecting substantially to the end of the tip orifice, or beyond the tip end if required to extract the IOL. Preferably, the tips contact each other in their fully compressed state. To provide the minimum bore size, and thereby maximum compression of the IOL, the tips preferably have parallel abutting faces and outer surfaces that have a curvature corresponding to the curvature of the bore wall. Since approximately the lower one-half of the tips are cut away, the tips in their fully extended position join to form a generally hemispherical cross-section, with each tip forming one-half of a hemisphere.

An alternative embodiment of the present invention relates to an IOL inserter that pulls the IOL from the inserter into the eye, as opposed to prior art inserters that discharge the IOL from the inserter by pushing on the rear of the IOL with a plunger. More specifically, the alternative embodiment differs from the preceding embodiment and the prior art in the plunger configuration and in a modification to the IOL that is inserted by the plunger.

This alternative embodiment is particularly adapted to insertion of intraocular lens comprised of an optic with hinged, preferably integrally formed, haptics in the form of plates having upper and lower surfaces. A representative IOL of the type contemplated by this embodiment of the present invention is sold under the trademark Crystalens™, manufactured by eyeonics, Inc., Aliso Viejo, Calif.

The IOL inserted by the alternative embodiment includes a plunger engagement point on the leading haptic, e.g., a projection or recess that is engaged by the plunger tip when the plunger is moved to its inserted position. For example, the IOL may include an axially aligned hole through the leading haptic.

The inserter used in connection with this alternative embodiment includes a plunger with distal end adapted to engage the engagement point on the leading haptic, so that the IOL is pulled through and out of the inserter as the plunger is moved to its inserted position. The plunger distal end may include, for example, an angled tip section, or a “Y” shaped tip with two opposed angled tip sections. The inserter is otherwise similar in construction to the previously described inserter, with the IOL being prepackaged in a cartridge held in a cartridge holder.

When used, the plunger is moved toward its extended position until the angled tip engages the hole in the leading haptic. Further movement of the plunger pulls the IOL into the conically tapered section of the inserter tip to compress the IOL to the desired cross-sectional diameter. Finally, the tip of the plunger is extended beyond the distal end of the inserter tip by a distance approximately equal to the length of the IOL to completely draw the IOL from the inserter tip into the eye, permitting the IOL to resume its uncompressed configuration. Preferably, the tip is angled forward at an angle of from about 30° to about 60° below horizontal, with the vertical dimension of the tip, i.e., the vertical distance between the top and bottom of the tip being up to about one-half of the cross-sectional diameter of the cartridge chamber.

Further details of the present invention will be apparent from the detailed description of the invention taken together with the drawings.

### BRIEF DESCRIPTION OF THE DRAWINGS

FIG. 1 is a top view of an inserter.

FIG. 2 is a sectional top view of a first inserter embodiment.

FIG. 3 is a sectional side view of the front part of the inserter of FIG. 2.

FIG. 4 is a sectional top view of a second inserter embodiment.

FIG. 5 is a sectional side view of the front part of the inserter of FIG. 3.

FIG. 6 is a sectional side view of an alternative nozzle with a grooved tapered bore.

FIG. 7 is a sectional end view of an alternative laterally compressible cartridge enclosing in IOL in the uncompressed state.

FIG. 8 is a sectional end view of an alternative laterally compressible cartridge enclosing in IOL in the compressed state.

### DETAILED DESCRIPTION OF THE INVENTION

In the following description, terms such as horizontal, upright, vertical, above, below, beneath, and the like, are used solely for the purpose of clarity in illustrating the invention, and should not be taken as words of limitation. The drawings are for the purpose of illustrating the invention and are not intended to be to scale.

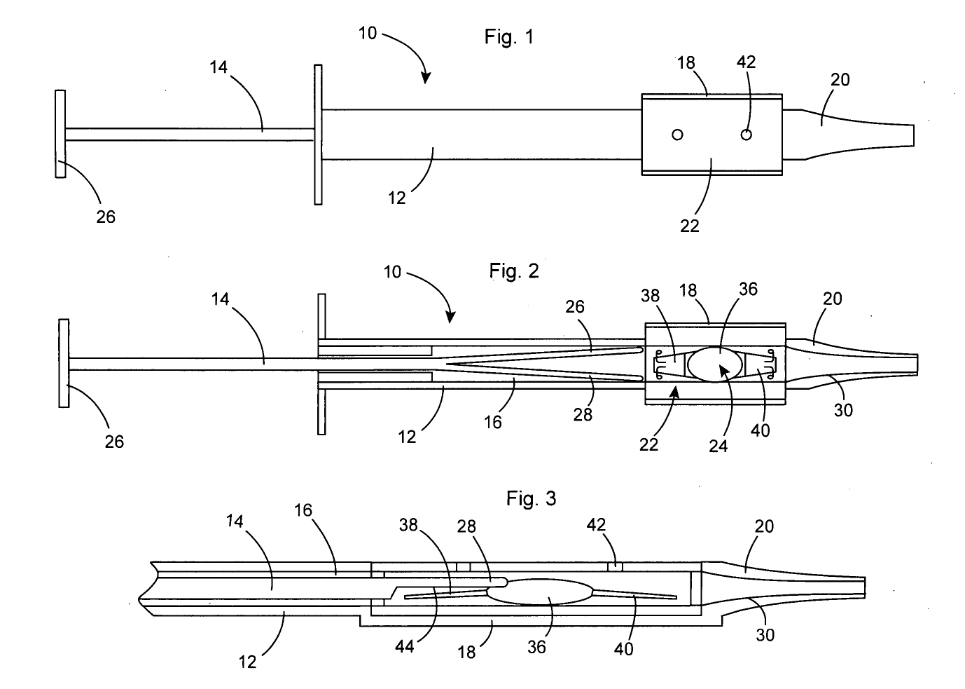

As best illustrated in FIGS. 1, 2 and 3, inserter, generally 10, is comprised of an elongated handpiece 12 having a proximal end and a distal end. Plunger 14 projects from the proximal end of handpiece 12 and is slidable within a bore 16 within handpiece 12. Cartridge holder 18 is attached to the distal end of handpiece 12, and discharge nozzle 20 is positioned at the distal end of holder 18. Cartridge 22, mounted in holder 18, encloses prepackaged IOL, generally 24.

Plunger 14 has a proximal end with an enlarged head 26 used by the surgeon to push plunger 14 forward from a retracted position to an inserted position. The distal end of plunger 14 is split or forked to form a pair of arms 26 and 28 that project toward the distal end of handpiece 12 with the tips of arms 26 and 28 being in contact with the wall of bore 16. Handpiece bore 16 terminates in a distal end of a given diameter at the proximal side of cartridge holder 18.

Discharge nozzle 20 includes a conical tapered bore 30 having a proximal end adjacent the distal side of cartridge holder 18, and a distal discharge opening 32. The longitudinal axis of conical bore 30 is aligned with the axis of handpiece bore 16, and the cross-sectional diameter of conical bore 30 at its proximal end is the same as the cross-sectional diameter of handpiece bore 16 at its distal end.

Prepackaged cartridge 22 is comprised of a housing 24 having an IOL chamber 34 enclosing IOL 24 with IOL optic 36 centrally positioned, and with haptics 38 and 40 on the trailing and leading sides of optic 36. The longitudinal axis of IOL 24 is aligned with the longitudinal axes of bores 16 and 30 when cartridge 22 is mounted in cartridge holder 18. Cartridge 22 has a proximal opening positionable adjacent the distal opening of bore 16 and a distal opening positionable adjacent the proximal opening of nozzle 20. Thus, bore 16, chamber 34 and bore 30 are axially aligned to form a continuous bore through inserter 10. Cartridge 22 also includes lubricant ports 42 to receive a suitable lubricant prior to IOL insertion.

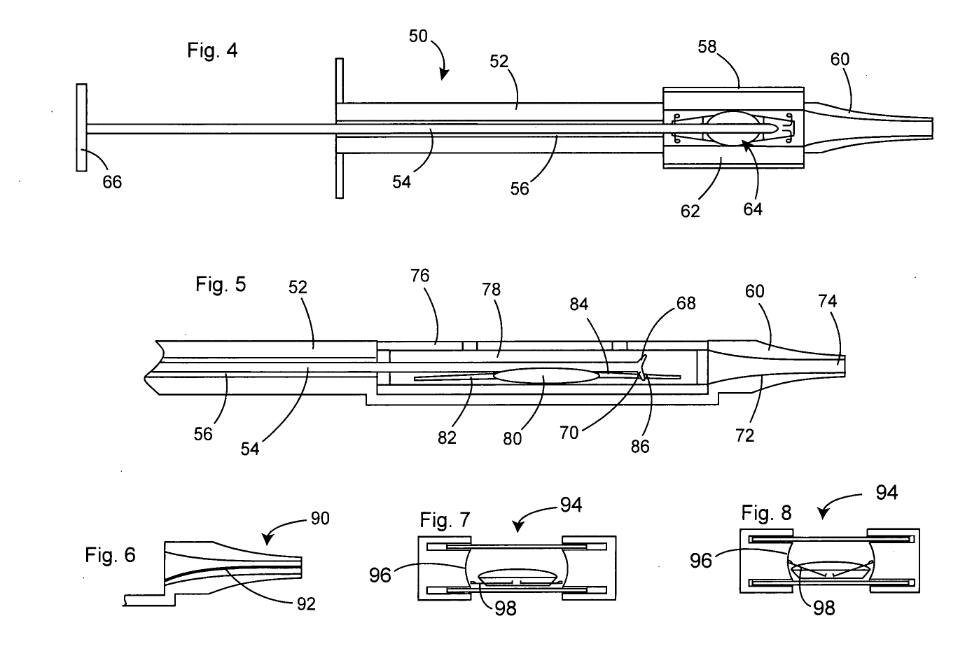

Arms 26 and 28 include cutout sections 44 on their lower sides adjacent their distal ends, so that arms 26 and 28 will pass above haptic 38 when plunger 14 is moved toward its extended position. Thus, as shown in FIG. 4, the tips of arms 26 and 28 push against the side edges of optic 36, instead of against haptic 38, minimizing the risk of damage to haptic 38.

Forward movement of plunger 14 pushes IOL 24 into nozzle bore 30 where the diminishing diameter of bore 30 results in lateral compression of IOL 24. In addition, the tips of arms 26 and 28 ride against the wall of bore 30 and are flexed inwardly by the curvature of the wall of bore 30, whereby the tips of arms 26 and 28 continue to engage the sides of optic 36. When plunger 14 reaches its fully inserted position, IOL 24 is discharged from discharge end 32 into the patient's eye, where IOL 24 expands to its original uncompressed configuration.

An alternative embodiment is illustrated in FIGS. 4 and 5. In this embodiment, inserter, generally 50 is comprised of an elongated handpiece 52 having a proximal end and a distal end, a plunger 54 projecting from the proximal end of handpiece 52 and slidable within bore 56 within handpiece 52. Cartridge holder 58 is attached to the distal end of handpiece 52, and a discharge nozzle 60 is positioned at the distal end of holder 58. Cartridge 62 holding a prepackaged IOL, generally 64, is shown positioned in holder 18.

Plunger 54 has a proximal end with an enlarged manipulation head 66. The distal end of plunger 54 has opposed angular IOL engagement projections 68 and 70 that extend forward and outward from plunger 54 at an angle of about 45° with the vertical distance of the tip being about one-half the cross-sectional diameter of the cartridge chamber.

Discharge nozzle 60 is constructed similar to nozzle 20 and includes a conical bore 72 having a proximal end adjacent the distal side of cartridge holder 58, and a distal discharge opening 74. The longitudinal axis of conical bore 72 is aligned with the axis of handpiece bore 56. Cartridge 62 is constructed similar to cartridge 22 and is comprised of a housing 76 having an IOL chamber 78 adapted to hold IOL 64 that is similar to IOL 24. Specifically, IOL 64 includes an optic 80 with haptics 82 and 84 on the trailing and leading sides of optic 80. In this embodiment, however, haptic 84 also includes an axially aligned hole 86 for receiving one of projections 68 or 70. The longitudinal axis of IOL 64 is aligned with the longitudinal axes of bores 56 and 70 when cartridge 62 is mounted in cartridge holder 58. Cartridge 58 includes a distal opening aligned with and equal in diameter to the proximal end of bore 72.

FIG. 6 illustrates an alternative nozzle, generally 90, that may be used instead of nozzle 20 or 60. Nozzle 90 differs from these nozzles in the inclusion of grooves 92 that incline upwardly from the proximal end to the distal end of nozzle 90, serving to guide the leading haptic of an IOL pushed through nozzle 90.

When used, plunger 54 is pushed toward its extended position, causing projections 68 and 70 to initially ride over the top of haptic 82 and optic 80. One of projections 68 or 70, depending on which projection is extending downwardly, is inserted into hole 86 in leading haptic 84. Plunger 54 can be rotated to align projection 68 or 70 with hole 86. Further forward movement of plunger 54 pulls IOL 64 into bore 72. As IOL 64 continues to move toward the distal end of bore 72, IOL 64 is laterally compressed to the desired insertion dimension. When in its fully extended position, the distal end of plunger 54 extends beyond the discharge end of nozzle 60, releasing IOL 64 from bore 72 and into the patient's eye, where IOL 64 regains its original configuration.

FIGS. 7 and 8 illustrate an alternative laterally compressible cartridge, generally 94, suitable for use with either of the embodiments described above, or with other inserters. Upon lateral compression from the uncompressed state shown in FIG. 7 to the compressed state shown in FIG. 8, curved side walls 96 force anchor wires 98 upwardly and inwardly over the haptic to minimize the risk of damage during insertion of the IOL.

Certain modifications and improvements will occur to those skilled in the art upon a reading of the foregoing description. It should be understood that all such modifications and improvements have been deleted herein for the sake of conciseness and readability but are properly within the scope of the following claims.

1. An intraocular lens inserter adapted to hold a prepackaged intraocular lens cartridge having a housing with a chamber containing an intraocular lens with an optic having sides, and trailing and leading haptics, said chamber having a continuous wall with opposed proximal and distal opening, said inserter comprising:
a) a handpiece having a handpiece bore with a continuous inner wall and a distal end;
b) a discharge nozzle spaced from the distal end of said handpiece, said nozzle having a tapered bore with a continuous inner wall, a proximal end and a discharge end;
c) a cartridge holder between said handpiece and said nozzle, said holder being adapted to hold said cartridge with said cartridge housing proximal opening aligned with said handpiece bore distal end, and said cartridge housing distal opening aligned with said nozzle proximal end; and
d) a plunger moveable between retracted and inserted positions, said plunger having a forked distal end with first and second opposed arms with distal ends extending forward and outwardly to sequentially engage said bore wall, said chamber wall and said nozzle tapered bore wall when said plunger is moved to its inserted position, whereby said arms maintain contact with the sides of the optic of an intraocular lens while said optic is laterally compressed by movement through said tapered nozzle.

2. The inserter of claim 1, wherein said arms have cutout lower sections extending rearwardly from the tips of said arms, whereby said arms extend above the trailing haptic of an intraocular lens within a cartridge mounted in said holder when said plunger is moved to its inserted position.

3. The inserter of claim 1, wherein the length of said arms is at least equal to the length of the trailing haptic of said intraocular lens.

4. The inserter of claim 1, wherein the distal end of said handpiece bore, said cartridge chamber and the proximal end of said nozzle bore have approximately the same cross-sectional shape.

5. The inserter of claim 1, wherein said nozzle tapered bore is grooved to guide said leading haptic.

6. An intraocular lens inserter comprising:
a) a handpiece having a handpiece bore with a continuous inner wall and a distal end;
b) a discharge nozzle spaced from the distal end of said handpiece, said nozzle having a tapered bore with a continuous inner wall, a proximal end and a discharge end;
c) a cartridge holder between said handpiece and said nozzle;
d) a prepackaged intraocular lens cartridge mounted in said holder, said cartridge including a housing with a chamber containing an intraocular lens with an optic having sides, and trailing and leading haptics, said chamber having a continuous wall with opposed proximal and distal opening, said holder being adapted to hold said cartridge with said cartridge housing proximal opening aligned with said handpiece bore distal end, and said cartridge housing distal opening aligned with said nozzle proximal end; and
e) a plunger moveable between retracted and inserted positions, said plunger having a forked distal end with first and second opposed arms with distal ends extending forward and outwardly to sequentially engage said bore wall, said chamber wall and said nozzle tapered bore wall when said plunger is moved to its inserted position, whereby said arms maintain contact with the sides of the optic of an intraocular lens while said optic is laterally compressed by movement through said tapered nozzle.

7. The inserter of claim 6, wherein said optic and said cartridge chamber have approximately the same lateral width.

8. The inserter of claim 6, wherein said intraocular lens is packaged within said cartridge in an uncompressed condition.

9. The inserter of claim 6, wherein said cartridge is laterally compressible.

10. The inserter of claim 6, wherein said cartridge includes lubricant addition ports.

11. The inserter of claim 6, wherein the length of said arms is at least equal to the length of the trailing haptic of said intraocular lens.

12. The inserter of claim 6, wherein the distal end of said handpiece bore, said cartridge chamber and the proximal end of said nozzle bore have approximately the same cross-sectional shape.

13. The inserter of claim 6, wherein said nozzle tapered bore is grooved to guide said leading haptic.

14. An intraocular lens inserter adapted to hold a prepackaged intraocular lens cartridge having a housing with a chamber containing an intraocular lens with an optic having a trailing haptic and a leading haptic with a plunger engagement point, said chamber having a continuous wall with opposed proximal and distal opening, said inserter comprising:
a) a handpiece having a handpiece bore with a continuous inner wall and a distal end;
b) a discharge nozzle spaced from the distal end of said handpiece, said nozzle having a tapered bore with a continuous inner wall, a proximal end and a discharge end;
c) a cartridge holder between said handpiece and said nozzle, said holder being adapted to hold said cartridge with said cartridge housing proximal opening aligned with said handpiece bore distal end, and said cartridge housing distal opening aligned with said nozzle proximal end; and
d) a plunger slidable within said bore between a retracted position and an extended position, said plunger having a distal end adapted to engage the engagement point of said leading haptic, said plunger pulling said intraocular lens through said tapered bore when said plunger is moved to its fully inserted position.

15. The inserter of claim 14, wherein said plunger has a distal end with an angled tip.

16. The inserter of claim 14, wherein said cartridge chamber has a given diameter, and the vertical length of said angular tip is about one-half of said chamber given diameter.

17. The inserter of claim 14, wherein said engagement point is an axially aligned hole in said leading haptic.

18. An intraocular lens cartridge comprising:
a) a housing having a lens chamber with proximal and distal openings, and a longitudinal axis extending through said openings; and
b) an intraocular lens within said chamber, said lens including an optic with a trailing haptic toward said proximal opening and a leading haptic toward said distal opening, said leading haptic including a plunger engagement point.

19. The cartridge of claim 18, wherein said housing includes lubricant ports into said chamber.

20. The cartridge of claim 18, wherein said optic and haptics are integrally molded from a compressible polymeric material.

21. The cartridge of claim 18, wherein said optic and said chamber have approximately the same lateral width.

22. The cartridge of claim 18, wherein said intraocular lens is in an uncompressed state.

23. The cartridge of claim 18, wherein said cartridge is laterally compressible.

24. An intraocular lens cartridge comprising:
a) a housing having a lens chamber with proximal and distal openings, a longitudinal axis extending through said openings, and lubricant ports into said chamber; and
b) an intraocular lens within said chamber, said lens including an optic with a trailing haptic toward said proximal opening and a leading haptic toward said distal opening.

25. The cartridge of claim 24, wherein said optic and haptics are integrally molded from a compressible polymeric material.

26. The cartridge of claim 24, wherein said optic and said chamber have approximately the same lateral width.

27. The cartridge of claim 24, wherein said cartridge is laterally compressible.
